(19) United States
(12) Patent Application Publication
(10) Pub. No.: US 2024/0200592 A1
(43) Pub. Date: Jun. 20, 2024

(54) SCREW-IN SWIVEL DEVICE

(71) Applicant: George Washington English, V, Charleston, SC (US)

(72) Inventor: George Washington English, V, Charleston, SC (US)

(21) Appl. No.: 18/066,795

(22) Filed: Dec. 15, 2022

(51) Int. Cl. F16C 11/04

(52) U.S. Cl. CPC F16C 11/04

(57) ABSTRACT

A swivel device includes a bearing member having a work piece end, a drive end, and first screw threads at the work piece end, the bearing member having a central axis extending between the work piece end and the drive end, an inner barrel fixed to the bearing member and having a first engagement feature, and an outer barrel around the bearing member and the inner barrel and including a second engagement feature, wherein the outer barrel is slidable along the central axis of the bearing member and in a first position the first engagement feature and the second engagement feature are disengaged and in a second position the first engagement feature and the second engagement feature are engaged fixing a rotation of the outer barrel relative to the bearing member and the inner barrel.

### BACKGROUND

The present disclosure relates generally to a swivel device and more particularly to a screw-in swivel device for controlling movement of a work piece.

When painting more than one side of a work piece, a painter will typically position the work piece on a flat surface, coat the exposed upper side of the work piece, allow the exposed upper side to dry, then flip the work piece and coat the second side. The complete job is delayed by at least the dry time of the side painted first.

### SUMMARY

In accordance with one or more embodiments of the present invention, a swivel device comprises: a bearing member comprising a work piece end, a drive end, and first screw threads at the work piece end, the bearing member having a central axis extending between the work piece end and the drive end; an inner barrel fixed to the bearing member and comprising a first engagement feature; and an outer barrel around the bearing member and the inner barrel and comprising a second engagement feature, wherein the outer barrel is slidable along the central axis of the bearing member and in a first position the first engagement feature and the second engagement feature are disengaged and in a second position the first engagement feature and the second engagement feature are engaged fixing a rotation of the outer barrel relative to the bearing member and the inner barrel.

In accordance with one or more embodiments of the present invention, a swivel device comprises: a lobed bearing member comprising a work piece end, a drive end, a flattened lobe between the work piece end and the drive end, and first screw threads at the work piece end, the bearing member having a central axis extending between the work piece end and the drive end, and an outer barrel around the bearing member and comprising a key barrel, wherein the outer barrel is slidable along the central axis of the bearing member and in a first position the flattened lobe and the key barrel are disengaged and in a second position the flattened lobe and the key barrel are engaged fixing a rotation of the outer barrel relative to the bearing member.

In accordance with one or more embodiments of the present invention, a swivel device comprises: a bearing member comprising a work piece end, a drive end, and first screw threads at the work piece end, the bearing member having a central axis extending between the work piece end and the drive end; an inner barrel fixed to the bearing member and comprising a first magnetic feature; and an outer barrel around the bearing member and the inner barrel and comprising a second magnetic feature, wherein the outer barrel is fixed along the central axis of the bearing member and capable of being rotated around the bearing member and the inner barrel, wherein the first magnetic feature and the second magnetic feature cooperate to hold a relative position of the outer barrel and the inner barrel.

### BRIEF DESCRIPTION OF THE DRAWINGS

The following drawings are presented by way of example only and without limitation, wherein like reference numerals (when used) indicate corresponding elements throughout the several views, and wherein:

FIG. 1 is a schematic illustration of an example swivel device, according to an embodiment of the present invention;

FIG. 2 is a schematic illustration of an example swivel device supporting a work piece, according to an embodiment of the present invention;

FIG. 3 is a schematic illustration of a trestle supporting an example swivel device, according to an embodiment of the present invention;

FIG. 4 is a schematic illustration of an example swivel device, according to an embodiment of the present invention;

FIG. 5 is a schematic illustration of an example swivel device, according to an embodiment of the present invention;

FIG. 6 is a schematic illustration of an example swivel device, according to an embodiment of the present invention;

FIG. 7A is a schematic illustration of an outer barrel of a swivel device, according to an embodiment of the present invention;

FIG. 7B is a schematic illustration of an inner barrel of a swivel device, according to an embodiment of the present invention;

FIG. 8A is a schematic illustration of an outer barrel of a swivel device, according to an embodiment of the present invention;

FIG. 8B is a schematic illustration of an inner barrel of a swivel device, according to an embodiment of the present invention;

FIG. 9A is a schematic illustration of a swivel device, according to an embodiment of the present invention;

FIG. 9B is a schematic illustration of the swivel device of FIG. 9A, according to an embodiment of the present invention;

FIG. 10 is a schematic illustration of an example swivel device, according to an embodiment of the present invention;

FIG. 11 is a schematic illustration of an example swivel device, according to an embodiment of the present invention;

FIG. 12 is a schematic illustration of a key barrel of a swivel device, according to an embodiment of the present invention;

FIG. 13 is a schematic illustration of a bearing member of a swivel device, according to an embodiment of the present invention; and

FIG. 14 is a schematic illustration of a swivel device, according to an embodiment of the present invention.

### DETAILED DESCRIPTION

Embodiments of the invention will be described herein in which a swivel device enables a work piece to be suspended from a trestle, commonly call a sawhorse, and rotated about a central axis, enabling access to multiple sides of the work piece with limited (or no) contact with the work piece. The swivel device may be used to rotate the work piece. The swivel device may include means to fix an orientation of the work piece around the central axis.

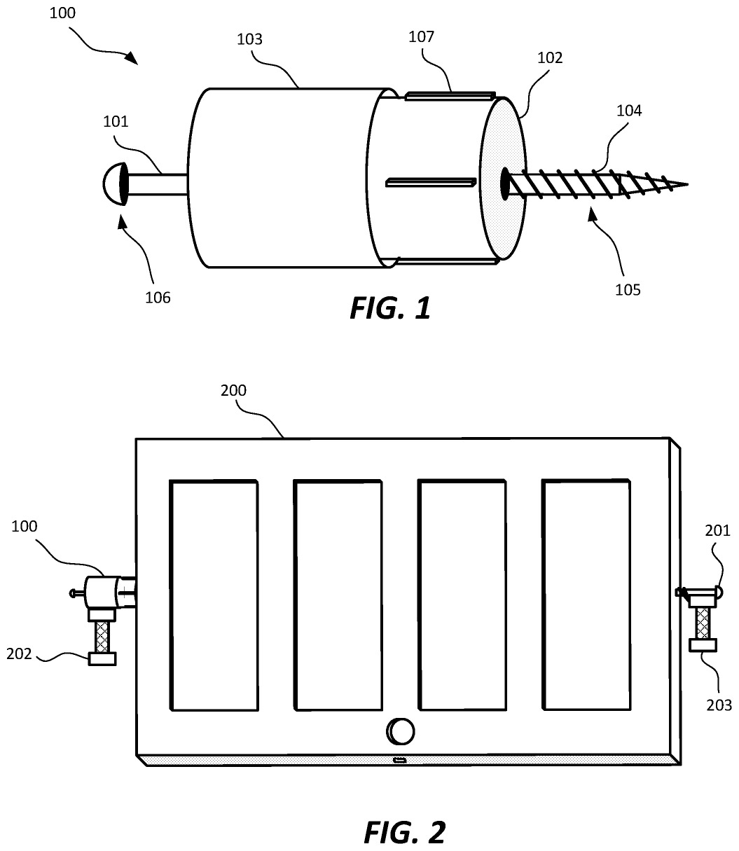

FIG. 1 is a schematic illustration of an example swivel device, according to an embodiment of the present invention. According to some embodiments, a swivel device 100 includes a bearing member 101, an inner barrel 102, and an outer barrel 103. The inner barrel 102 is fixed to the bearing member. For example, the inner barrel 102 may be bonded to, welded to, and/or screwed onto the bearing member 101. The outer barrel 103 is free to rotate on the central axis of the bearing member 101. The bearing member 101 includes screw threads 104 at a work piece end 105, and may include a screw drive at a drive end 106. The screw drive may be a Phillips head drive, a slotted head drive, a hex head drive, or another means for driving the bearing member 101 into a work piece.

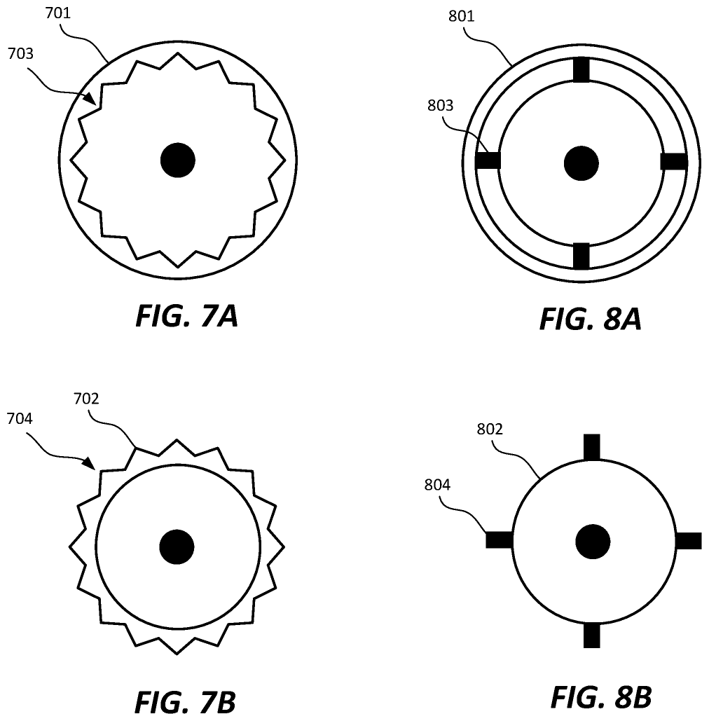

In some embodiments, each of the inner barrel 102 and the outer barrel 103 include cooperating engagement features. As illustrated in FIG. 1, the inner barrel 102 includes one or more first keys 107, which may fit in cooperating slots on an inner surface of the outer barrel 103. See for example, FIG. 7A and FIG. 8A.

In some embodiments, the engagement features of the inner barrel 102 and the outer barrel 103 may be disengaged by sliding the inner barrel 102 partially apart from the outer barrel 103 as illustrated in FIG. 1. Once the engagement features are disengaged, the inner barrel 102 may be turned on the central axis inside of the outer barrel 103.

FIG. 2 is a schematic illustration of an example swivel device supporting a work piece, according to an embodiment of the present invention. In the example, the work piece is a door. The swivel device 100 may be driven into an end surface of a work piece 200. A screw 201 is driven into an opposite end of the work piece 200. The swivel device 100 and the screw 201, driven into the work piece 200, may be placed on a first trestle 202 and a second trestle 203, respectively.

According to some aspects, when the swivel device 100 is driven into a surface of the work piece 200, an orientation of the inner barrel 102 is fixed relative to the work piece. In some aspects, the work piece 200 can rotate relative to a central axis of the outer barrel 103.

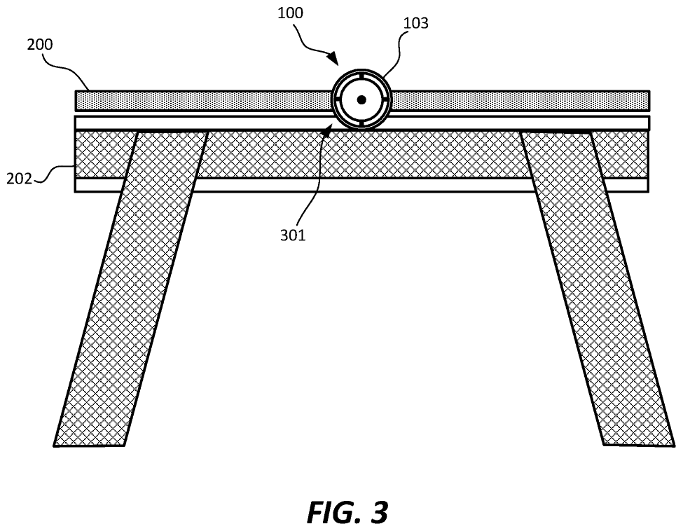

FIG. 3 is a schematic illustration of the first trestle 202, according to an embodiment of the present invention. In some aspects, the first trestle 202 may include a notch 301 to receive the outer barrel 103 of the swivel device 100. In some aspects, the notch 301 may be a recess in a surface of a support structure, In other aspects, the notch 301 may be two blocks attached to a surface of a support structure and having a gap therebetween, or a single block attached to a surface of a support and having a recess therein. In some aspects, the notch 301 may fix an orientation and a lateral position of the outer barrel 103 relative to the first trestle 202, for example, at least due to a weight of the work piece on the first trestle 202 through the swivel device 100. The second trestle 203 may be used to support the screw 201 and may also include a notch. According to some embodiments, the notches may be omitted. It should be understood that the trestle can be any support, such as a pair of tables, a rack, or other support structure.

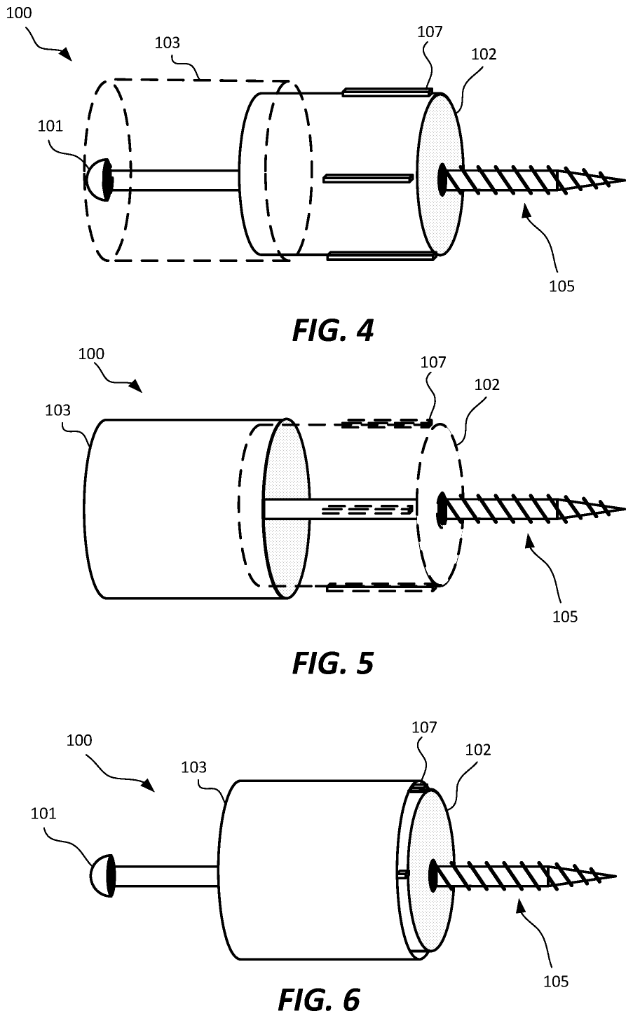

FIG. 4 and FIG. 5 are schematic illustrations of the swivel device 100, according to an embodiment of the present invention. According to some aspects, the inner barrel 102 has a smaller diameter than the outer barrel 103. According to at least one aspect, a portion of the inner barrel 102 and the outer barrel 103 overlap even in the case that the engagement features are disengaged.

As illustrated in FIG. 6, the engagement features are engaged when the inner barrel 102 is moved within the outer barrel 103. In a case that the inner barrel 102 is moved within the outer barrel 103, a relative orientation of the inner barrel 102 and the outer barrel 103 around the central axis of the bearing member 101 is locked.

FIG. 7A is a schematic illustration of a shaped outer barrel 701 of a swivel device, according to an embodiment of the present invention. FIG. 7B is a schematic illustration of a shaped inner barrel 702 of a swivel device, according to an embodiment of the present invention. The shaped inner barrel 702 fits within the shaped outer barrel 701.

According to some embodiments, the shaped outer barrel 701 includes first shaped features 703. The shaped inner barrel 702 includes second shaped features 704. In one example, the shaped features are a number of peaks, for example, 16 peaks. According to some aspects, the shaped features may be engaged by sliding the shaped outer barrel 701 and the shaped inner barrel 702 together, along their central axes, where the first shaped features 703 and the second shaped features 704 are made to overlap. According to some aspects, the shaped features may be disengaged by sliding the shaped outer barrel 701 and the shaped inner barrel 702 apart, along their central axes, until the first shaped features 703 and the second shaped features 704 are separated.

According to some aspects, once the shaped features are disengaged, the shaped outer barrel 701 and the shaped inner barrel 702 may be rotated relative to one another. It follows that the work piece, fixed to the shaped inner barrel 702 may be rotated relative to the shaped outer barrel 701.

FIG. 8A is a schematic illustration of a keyed outer barrel 801 of a swivel device, according to an embodiment of the present invention. FIG. 8B is a schematic illustration of a keyed inner barrel 802 of a swivel device, according to an embodiment of the present invention. The keyed inner barrel 802 fits within the keyed outer barrel 801.

According to some embodiments, the keyed outer barrel 801 includes one or more first key features 803. The keyed inner barrel 802 includes one or more second keyed features 804. In one example, the keyed features include keys and slots. As illustrated the keyed inner barrel 802 includes 4 keys and the keyed outer barrel 801 includes 4 slots, matching the keys.

According to some aspects, the keyed features may be engaged by sliding the keyed outer barrel 801 and the keyed inner barrel 802 together, where the first keyed features 803 and the second keyed features 804 are made to overlap. According to some aspects, the keyed features may be disengaged by sliding the keyed outer barrel 801 and the keyed inner barrel 802 apart until the first keyed features 803 and the second keyed features 804 are separated.

According to some aspects, once the keyed features are disengaged, the keyed outer barrel 801 and the keyed inner barrel 802 may be rotated relative to one another. It follows that the work piece, fixed to the keyed inner barrel 802 may be rotated relative to the keyed outer barrel 801.

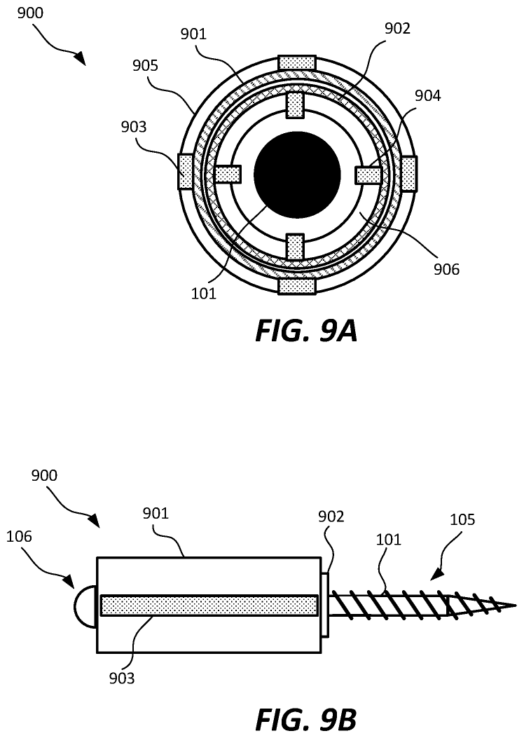

FIG. 9A is a schematic illustration of a magnetic swivel device 900, according to an embodiment of the present invention. FIG. 9B is a schematic illustration of the magnetic swivel device 900 of FIG. 9A, according to an embodiment of the present invention. The magnetic swivel device 900 includes a magnet augmented outer barrel 901 and a magnet augmented inner barrel 902, according to an embodiment of the present invention. The magnet augmented inner barrel 902 fits within the magnet augmented outer barrel 901.

According to some embodiments, the magnet augmented outer barrel 901 includes one or more first magnetic features 903. The magnet augmented inner barrel 902 includes one or more second magnetic features 904. According to some aspects, the first and/or the second magnetic features may be bonded to the respective barrel by an adhesive and/or fit into blocking (e.g., outer blocking 905 and inner blocking 906) on a surface of the barrel to secure a position of the magnetic features.

In one example, the magnetic features are disposed such that the first magnetic features 903 and the second magnetic features 904 are attracted to one another. That is, the poles of the magnetic features on one of the barrels are reversed on the other barrel. As illustrated the magnet augmented inner barrel 902 includes 4 magnetic features and the magnet augmented outer barrel 901 includes 4 magnetic features.

According to some aspects, the magnetic features of the magnet augmented outer barrel 901 and the magnet augmented inner barrel 902 are maintained in alignment by the magnetic force. The magnetic force may be overcome by applying a force to the work piece to rotate the inner magnet augmented inner barrel 902, for example, to a point where the magnetic features are again in alignment, e.g., 90 degrees from a starting point.

In some embodiments, the inner barrel and the outer barrel do not slide with respect to one another's central axis. In one example, the magnet augmented inner barrel 902 is fixed to the screw drive and the magnet augmented outer barrel 901 can be rotated around the magnet augmented inner barrel 902 while being held in place along the central axis by a head of the screw drive at the drive end 106. According to some aspects, the magnet augmented outer barrel 901 may be held in place along the central axis by a C-clip, and O-ring, the head of the screw drive, for example.

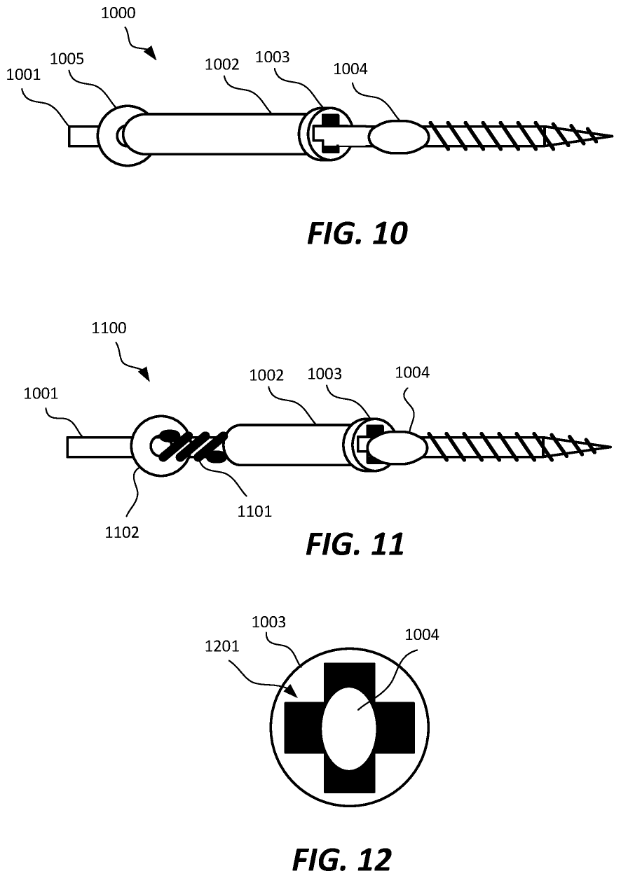

FIG. 10 is a schematic illustration of an example swivel device, according to an embodiment of the present invention. In some aspects, an inner barrel may be omitted. For example, as illustrated in FIG. 10, a second swivel device 1000 includes a lobed bearing member 1001 and a barrel 1002 around the lobed bearing member 1001. In at least one configuration the barrel 1002 may rotate around the central axis of the lobed bearing member 1001. A key barrel 1003 is disposed at an end of the barrel 1002. In some embodiments, the key barrel 1003 may be joined to the barrel 1002, for example, by a weld. According to some embodiments, the key barrel 1003 and the barrel 1002 are a single piece, for example, where a shape of the key barrel 1003 is milled in the barrel 1002.

According to some aspects, the lobed bearing member 1001 includes a flattened lobe 1004 in a middle portion of the lobed bearing member 1001. According to some aspects, the key barrel 1003 may be engaged with the flattened lobe 1004, fixing the key barrel 1003 and the barrel 1002 to the lobed bearing member 1001 and a connected work piece (not shown). A position of the key barrel 1003 and the barrel 1002 along the central axis of the lobed bearing member 1001 may be set by a fixing member 1005 such as a C-clip around a grove in the lobed bearing member 1001 or a hitch pin clip through a hole in the lobed bearing member 1001.

FIG. 11 is a schematic illustration of an example swivel device, according to an embodiment of the present invention. According to some aspects, a second swivel device 1100 includes the lobed bearing member 1001 and the barrel 1002 around the bearing member 1001. In at least one configuration the barrel 1002 may rotate around the bearing member 1001. The key barrel 1003 is disposed at an end of the barrel 1002. In some embodiments, the key barrel 1003 may be joined to the barrel 1002, for example, by a weld. According to some embodiments, the key barrel 1003 and the barrel 1002 are a single piece, for example, where a shape of the key barrel 1003 is milled in the barrel 1001.

According to some aspects, the bearing member 1001 includes a flattened lobe 1004 in a middle portion of the bearing member 1001. According to some aspects, the key barrel 1003 may be engaged with the flattened lobe 1004, fixing the key barrel 1003 and the barrel 1002 to the bearing member 1001 and a connected work piece (not shown). A position of the key barrel 1003 and the barrel 1002 along the central axis of the bearing member 1001 may be set by a tensioning member 1101 and a fixed member 1102 such as an O-clip press fit onto the bearing member 1001. The tensioning member 1101 enables the key barrel 1003 and the barrel 1002 to be disengaged from the flattened lobe 1004 and the bearing member 1001 to be rotated within the key barrel 1003 and the barrel 1002.

FIG. 12 is a schematic illustration of the key barrel 1003, according to an embodiment of the present invention. The key barrel 1003 may include two or more keys 1201 milled into an inside surface thereof.

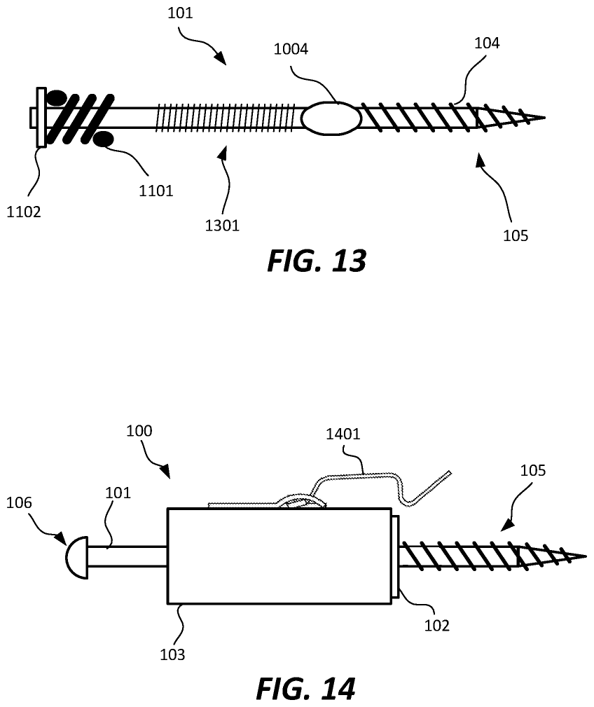

FIG. 13 is a schematic illustration of a bearing member of a swivel device, according to an embodiment of the present invention. The bearing member 101 includes first screw threads 104 at a work piece end 105. The bearing member 101 may include the flattened lobe 1004 in a middle portion of the bearing member 101 and second screw threads 1301 away from the first screw threads 104. The second screw threads may enable an inner barrel to be connected to the bearing member 101. According to at least one embodiments, the first screw threads 104 may be right-hand threads and the second screw threads 1301 may be left-hand threads. Accordingly, the inner barrel may be tightened on the bearing member 101 as the bearing member 101 is driven into a work piece. In some aspects, the first screw threads 104 may be wood-type threads and the second screw threads 1301 may be metal-type threads.

According to some embodiments, the bearing member 101 (e.g., as illustrated in FIG. 1, FIG. 6) may include the tensioning member 1101 and a fixed member 1102 such as an O-clip press fit onto the lobed bearing member 1001. In some aspects, the O-clip is press for onto a grove in the lobed bearing member 1001. According to some embodiments, the tensioning member 1101 enables an outer barrel to be maintained in an engaged position with an inner barrel. The inner barrel may be disengaged from the outer barrel by pulling the barrels apart, which compresses the tensioning member 1101, whereupon the inner barrel may be rotated relative to the outer barrel.

According to some embodiments and referring to FIG. 14, the outer barrel 103 may include a securing member 1401, such as a latch with a spring clip or a draw latch. For example, in the case of the spring clip, the outer barrel 103 and the inner barrel 102 may be engaged, where the inner barrel 102 is held in position by the securing member 1401.

### Recapitulation:

According to some embodiments, a swivel device comprises: a bearing member comprising a work piece end, a drive end, and first screw threads at the work piece end, the bearing member having a central axis extending between the work piece end and the drive end; an inner barrel fixed to the bearing member and comprising a first engagement feature; and an outer barrel around the bearing member and the inner barrel and comprising a second engagement feature, wherein the outer barrel is slidable along the central axis of the bearing member and in a first position the first engagement feature and the second engagement feature are disengaged and in a second position the first engagement feature and the second engagement feature are engaged fixing a rotation of the outer barrel relative to the bearing member and the inner barrel.

According to some aspects, the inner barrel and the outer barrel partially overlap when the outer barrel is in the first position.

According to some aspects, the first engagement feature and the second engagement feature are shaped features. According to some aspects, the first engagement feature and the second engagement feature are keyed features.

According to some aspects, the swivel device includes at least one first engagement feature and at least two second engagement features. According to some aspects, the swivel device includes at least two first engagement features and at least one second engagement feature.

According to some aspects, the bearing member comprises second screw threads in a middle portion of the bearing member, wherein the inner barrel is screwed onto the second screw threads. According to some aspects, an orientation of the first threads is opposite to an orientation of the second threads.

According to some aspects, the swivel device further comprises a fixed member at a drive end of the bearing member, and a tensioning member disposed on the bearing member between the fixed member and an end of the outer barrel. According to some aspects, the tensioning member is relatively compressed when the outer barrel is in the first position and relatively relaxed when the outer barrel is in the second position.

According to some aspects, the swivel device comprises a securing member mounted to an outer surface of the outer barrel, wherein the securing member has an open position and a closed position, and in the closed position the outer barrel is secured in the second position in which the first engagement feature and the second engagement feature are engaged fixing the rotation of the outer barrel relative to the bearing member and the inner barrel.

According to an embodiment, a swivel device comprises: a lobed bearing member comprising a work piece end, a drive end, a flattened lobe between the work piece end and the drive end, and first screw threads at the work piece end, the bearing member having a central axis extending between the work piece end and the drive end; and an outer barrel around the bearing member and comprising a key barrel, wherein the outer barrel is slidable along the central axis of the bearing member and in a first position the flattened lobe and the key barrel are disengaged and in a second position the flattened lobe and the key barrel are engaged fixing a rotation of the outer barrel relative to the bearing member.

According to some aspects, the key barrel comprises two or more key on an inside surface thereof.

According to some aspects, the swivel device further comprises: a fixed member at a drive end of the bearing member; and a tensioning member disposed on the bearing member between the fixed member and an end of the outer barrel. According to some aspects, the tensioning member is relatively compressed when the outer barrel is in the first position and relatively relaxed when the outer barrel is in the second position.

According to an embodiment, a swivel device comprises: a bearing member comprising a work piece end, a drive end, and first screw threads at the work piece end, the bearing member having a central axis extending between the work piece end and the drive end; an inner barrel fixed to the bearing member and comprising a first magnetic feature; and an outer barrel around the bearing member and the inner barrel and comprising a second magnetic feature, wherein the outer barrel is fixed along the central axis of the bearing member and capable of being rotated around the bearing member and the inner barrel, wherein the first magnetic feature and the second magnetic feature cooperate to hold a relative position of the outer barrel and the inner barrel.

According to some aspects, the swivel device comprises at least two of the first magnetic features and at least two of the second magnetic features.

According to some aspects, the bearing member comprises second screw threads in a middle portion of the bearing member, wherein the inner barrel is screwed onto the second screw threads. According to some aspects, an orientation of the first threads is opposite to an orientation of the second threads.

The illustrations of embodiments of the invention described herein are intended to provide a general understanding of the structure of various embodiments, and they are not intended to serve as a complete description of all the elements and features of apparatus and systems that might make use of the structures described herein. Many other embodiments will become apparent to those skilled in the art given the teachings herein; other embodiments are utilized and derived therefrom, such that structural and logical substitutions and changes can be made without departing from the scope of this disclosure. The drawings are also merely representational and are not drawn to scale. Accordingly, the specification and drawings are to be regarded in an illustrative rather than a restrictive sense.

Embodiments of the invention are referred to herein, individually and/or collectively, by the term “embodiment” merely for convenience and without intending to limit the scope of this application to any single embodiment or inventive concept if more than one is, in fact, shown. Thus, although specific embodiments have been illustrated and described herein, it should be understood that an arrangement achieving the same purpose can be substituted for the specific embodiment(s) shown; that is, this disclosure is intended to cover any and all adaptations or variations of various embodiments. Combinations of the above embodiments, and other embodiments not specifically described herein, will become apparent to those of skill in the art given the teachings herein.

The abstract is provided to comply with 37 C.F.R. § 1.72(b), which requires an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the appended claims reflect, inventive subject matter lies in less than all features of a single embodiment. Thus the following claims are hereby incorporated into the Detailed Description, with each claim standing on its own as separately claimed subject matter.

Given the teachings of embodiments of the invention provided herein, one of ordinary skill in the art will be able to contemplate other implementations and applications of the techniques of embodiments of the invention. Although illustrative embodiments of the invention have been described herein with reference to the accompanying drawings, it is to be understood that embodiments of the invention are not limited to those precise embodiments, and that various other changes and modifications are made therein by one skilled in the art without departing from the scope of the appended claims.

What is claimed is:

1. A swivel device comprising:
a bearing member comprising a work piece end, a drive end, and first screw threads at the work piece end, the bearing member having a central axis extending between the work piece end and the drive end;
an inner barrel fixed to the bearing member and comprising a first engagement feature; and
an outer barrel around the bearing member and the inner barrel and comprising a second engagement feature, wherein the outer barrel is slidable along the central axis of the bearing member and in a first position the first engagement feature and the second engagement feature are disengaged and in a second position the first engagement feature and the second engagement feature are engaged fixing a rotation of the outer barrel relative to the bearing member and the inner barrel.

2. The swivel device of claim 1, wherein the inner barrel and the outer barrel partially overlap when the outer barrel is in the first position.

3. The swivel device of claim 1, wherein the first engagement feature and the second engagement feature are shaped features.

4. The swivel device of claim 1, wherein the first engagement feature and the second engagement feature are keyed features.

5. The swivel device of claim 1, comprising at least one first engagement feature and at least two second engagement features.

6. The swivel device of claim 1, comprising at least two first engagement features and at least one second engagement feature.

7. The swivel device of claim 1, wherein the bearing member comprises second screw threads in a middle portion of the bearing member, wherein the inner barrel is screwed onto the second screw threads.

8. The swivel device of claim 7, wherein an orientation of the first screw threads is opposite to an orientation of the second screw threads.

9. The swivel device of claim 1, further comprising:
a fixed member at a drive end of the bearing member; and
a tensioning member disposed on the bearing member between the fixed member and an end of the outer barrel.

10. The swivel device of claim 9, wherein the tensioning member is relatively compressed when the outer barrel is in the first position and relatively relaxed when the outer barrel is in the second position.

11. The swivel device of claim 1, further comprising a securing member mounted to an outer surface of the outer barrel, wherein the securing member has an open position and a closed position, and in the closed position the outer barrel is secured in the second position in which the first engagement feature and the second engagement feature are engaged fixing the rotation of the outer barrel relative to the bearing member and the inner barrel.

12. A swivel device comprising:
a lobed bearing member comprising a work piece end, a drive end, a flattened lobe between the work piece end and the drive end, and first screw threads at the work piece end, the bearing member having a central axis extending between the work piece end and the drive end; and
an outer barrel around the bearing member and comprising a key barrel, wherein the outer barrel is slidable along the central axis of the bearing member and in a first position the flattened lobe and the key barrel are disengaged and in a second position the flattened lobe and the key barrel are engaged fixing a rotation of the outer barrel relative to the bearing member.

13. The swivel device of claim 12, wherein the key barrel comprises two or more key on an inside surface thereof.

14. The swivel device of claim 12, further comprising:
a fixed member at a drive end of the bearing member; and
a tensioning member disposed on the bearing member between the fixed member and an end of the outer barrel.

15. The swivel device of claim 14, wherein the tensioning member is relatively compressed when the outer barrel is in the first position and relatively relaxed when the outer barrel is in the second position.

16. A swivel device comprising:
a bearing member comprising a work piece end, a drive end, and first screw threads at the work piece end, the bearing member having a central axis extending between the work piece end and the drive end;
an inner barrel fixed to the bearing member and comprising a first magnetic feature; and
an outer barrel around the bearing member and the inner barrel and comprising a second magnetic feature, wherein the outer barrel is fixed along the central axis of the bearing member and capable of being rotated around the bearing member and the inner barrel, wherein the first magnetic feature and the second magnetic feature cooperate to hold a relative position of the outer barrel and the inner barrel.

17. The swivel device of claim 16, comprising at least two of the first magnetic features and at least two of the second magnetic features.

18. The swivel device of claim 16, wherein the bearing member comprises second screw threads in a middle portion of the bearing member, wherein the inner barrel is screwed onto the second screw threads.

19. The swivel device of claim 18, wherein an orientation of the first screw threads is opposite to an orientation of the second screw threads.
